(19) United States
(12) Patent Application Publication
(10) Pub. No.: US 2003/0226159 A1
(43) Pub. Date: Dec. 4, 2003

(54) Cancer models

(76) Inventors: Robert M. Bachoo, Roslindale, MA (US); Ronald A. Depinho, Brookline, MA (US)

Correspondence Address: FISH & RICHARDSON PC, 225 FRANKLIN ST, BOSTON, MA, 02110, US

(21) Appl. No.: 10/414,460

(22) Filed: Apr. 15, 2003

Related U.S. Application Data
(60) Provisional application No. 60/373,139, filed on Apr. 16, 2002.
(60) Provisional application No. 60/374,791, filed on Apr. 22, 2002.

(51) Int. Cl. A01K 67/027; C12N 5/06

(52) U.S. Cl. 800/18; 435/354

(57) ABSTRACT

The invention provides chimeric non-human animals, methods for making and using chimeric non-human animals, isolated stem cells, and methods for identifying agents that reduces cancer in a non-human animal. For example, the invention relates to using stem cells to make chimeric non-human animals having cancer or the ability to develop cancer. Such animals can be used to evaluate tumorigenesis, tumor maintenance, and tumor regression in vivo. In addition, the chimeric non-human animals provided herein can be used to identify agents that reduce or prevent tumor formation or growth in vivo.

### RELATED APPLICATIONS

This application claims the benefit of U.S. Provisional Application Serial No. 60/373,139, filed Apr. 16, 2002 and U.S. Provisional Application Serial No. 60/374,791, filed Apr. 22, 2002.

### BACKGROUND

1. Technical Field

The invention relates to methods and materials involved in making and using animal models. Specifically, the invention relates to using stem cells to make chimeric non-human animals having tumors or the ability to develop tumors.

2. Background Information

Transgenic and knock-out technology involves producing an animal where the germline of that animal has been genetically altered. Such technology has had a significant impact on all areas of the biological sciences. For example, transgenic mice have been used extensively in such areas as immunology, oncology, and neurobiology to study the roles particular polypeptides play in immunity, cancer development, and brain function. Likewise, knock-out mice have been instrumental to identifying the function of numerous polypeptides.

In addition, many animal models of disease have been made using transgenic or knock-out technology. For example, oncogenes have been overexpressed in mice using standard transgenic technology to produce mouse models for cancer. Such mouse models have been used to better understand not only cancer development and regression but also cancer treatment. In fact, many potential cancer treatments are initially identified using cancer models produced using either transgenic or knock-out technology.

### SUMMARY

The invention provides chimeric non-human animals, methods for making and using chimeric non-human animals, isolated stem cells, and methods for identifying agents that reduce cancer in a non-human animal. In particular, the invention relates to using stem cells to make chimeric non-human animals having cancer or the ability to develop cancer. Such animals can be used to study cancer and cancer treatment in vivo. For example, the chimeric non-human animals provided herein can be used to evaluate tumorigenesis, tumor maintenance, and tumor regression. In addition, the chimeric non-human animals provided herein can be used to identify agents that reduce or prevent tumor formation or growth in vivo.

The invention is based on the discovery that isolated stem cells can be genetically engineered such that when they are implanted into a non-human animal they not only integrate into the animal's tissue and differentiate into specific cell types but they also have the ability to form tumor cells. The tumor cells arising from implanted stem cells, or the differentiated cells originating from implanted stem cells, can be present within the animal's tissue in an integrated manner as opposed to being an isolated collection of tumor cells that typically results from injecting cultured tumor cells into a particular tissue. In addition, the chimeric non-human animals provided herein are unique cancer models in that the tumor cells arising from implanted stem cells, or the differentiated cells originating from implanted stem cells, can arise from cells that have been functionally integrated into the animal. For example, a mouse implanted with human neural stem cells engineered to be tumorigenic can develop human astrocytes that are integrated into the mouse's brain. While in this tissue environment, one or more of these astrocytes can develop into tumor cells. Thus, cancer progression within these models can be more similar to the events that lead to cancer development in nature than the types of cancer progression that occur when, for example, tumorigenic cells that are unable to integrate into the recipient's tissue are used.

In general, the invention features a non-human animal containing tumor cells originating, in the animal, from at least one heterologous stem cell, wherein the at least one heterologous stem cell contains a genetic alteration. The animal can be a rodent (e.g., mouse or rat). The animal can be immunocompromised. For example, the animal can be a SCID mouse. The at least one heterologous stem cell can be a mouse cell or a human cell. The at least one heterologous stem cell can be a neural stem cell, an embryonic stem cell, or a tissue-specific stem cell. The at least one heterologous stem cell can be a dedifferentiated cell (e.g., a dedifferentiated astrocyte, dedifferentiated hepatocyte, or dedifferentiated pancreatic cell). The dedifferentiated cell can be produced from a cultured cell (e.g., astrocytes, melanocytes, pancreatic cells, mammary epithelial cells, or hepatocytes). The genetic alteration can contain an introduced nucleic acid molecule. The introduced nucleic acid molecule can encode a polypeptide. The polypeptide can be an oncogene product. The polypeptide can be H-RAS, epidermal growth factor receptor, ErbB2, or MDM2. The polypeptide can be expressed by the at least one heterologous stem cell. The genetic alteration can contain a mutation. The mutation can be in a tumor suppressor gene. The tumor suppressor gene can encode p16^{INK4a}, p19^{ARF}, PTEN, or pRB. The genetic alteration can further contain an introduced nucleic acid molecule. The at least one heterologous stem cell can have reduced expression of the tumor suppressor gene. The at least one heterologous stem cell can have reduced tumor suppressor activity. The tumor cell can be a pancreatic tumor cell, a liver tumor cell, a breast tumor cell, or a brain tumor cell.

In another embodiment, the invention features an isolated stem cell, wherein the isolated stem cell is INK4a/ARF^{-/-}. The isolated stem cell can be isolated from brain, pancreas, or liver tissue. The isolated stem cell can be a dedifferentiated cell (e.g., a dedifferentiated astrocyte, dedifferentiated melanocyte, dedifferentiated hepatocyte, or dedifferentiated pancreatic cell). The dedifferentiated cell can be produced from a cultured cell (e.g., astrocytes, melanocytes, hepatocytes, mammary epithelial cells, or pancreatic cells).

Another embodiment of the invention features a method for making a chimeric non-human animal. The method includes (a) genetically altering stem cells, and (b) introducing the stem cells into a non-human animal thereby forming the chimeric non-human animal, wherein the chimeric non-human animal develops tumor cells from at least one of the stem cells. The chimeric non-human animal can be a rodent (e.g., mouse or rat). The stem cells can be mouse cells or human cells. The stem cells can be embryonic stem cells, tissue-specific stem cells, or dedifferentiated cells. Step (a) can include introducing a nucleic acid molecule into the stem cells. The nucleic acid molecule can encode a polypeptide. The polypeptide can be an oncogene product. Step (a) can include introducing a mutation into the stem cells. The mutation can be in a tumor suppressor gene. The mutation can reduce the expression of the tumor suppressor gene within the stem cells. The tumor cells can be pancreatic tumor cells, liver tumor cells, a breast tumor cell, or brain tumor cells.

Another embodiment of the invention features a method for making a cancer model. The method includes (a) genetically altering stem cells to have reduced tumor suppressor gene expression or increased oncogene expression, (b) introducing the stem cells into at least one non-human animals, and (c) identifying a cancerous non-human animal from the at least one non-human animals, wherein the cancerous non-human animal contains a tumor cell originating from at least one of the introduced stem cells, the cancerous non-human animal being the cancer model. The cancer model can be a mouse. The stem cells can be mouse cells or human cells. The stem cells can be embryonic stem cells, tissue-specific stem cells, or dedifferentiated cells. Step (a) can be performed in vitro. The tumor cell can be a pancreatic tumor cell, a liver tumor cell, a breast tumor cell, or a brain tumor cell.

In another embodiment, the invention features a non-human animal containing at least one heterologous stem cell, wherein the at least one heterologous stem cell is tumorigenic. The animal can be a rodent (e.g., rat or mouse). The animal can be immunocompromised. The animal can be a SCID mouse. The at least one heterologous stem cell can be a mouse cell or a human cell. The at least one heterologous stem cell can be a neural stem cell, an embryonic stem cell, or tissue-specific stem cell. The at least one heterologous stem cell can be a dedifferentiated cell (e.g., a dedifferentiated astrocyte, dedifferentiated hepatocyte, or dedifferentiated pancreatic cell). The dedifferentiated cell can be produced from a cultured cell (e.g., astrocytes, melanocytes, pancreatic cells, mammary epithelial cells, or hepatocytes). The at least one heterologous stem cell can be slightly tumorigenic, moderately tumorigenic, or highly tumorigenic. The at least one heterologous stem cell can contain a genetic alteration. The genetic alteration can contain an introduced nucleic acid molecule. The introduced nucleic acid molecule can encode a polypeptide. The polypeptide can be an oncogene product. The polypeptide can be H-RAS, epidermal growth factor receptor, ErbB2, or MDM2. The polypeptide can be expressed by the at least one heterologous stem cell. The genetic alteration can contain a mutation. The mutation can be in a tumor suppressor gene. The tumor suppressor gene can encode a polypeptide such as p16^{INK4a}, p19^{ARF}, PTEN, or pRB. The genetic alteration can further contain an introduced nucleic acid molecule. The at least one heterologous stem cell can have reduced expression of the tumor suppressor gene. The at least one heterologous stem cell can have reduced tumor suppressor activity. The animal can contain a tumor cell originating, in the animal, from the at least one heterologous stem cell. The tumor cell can be a pancreatic tumor cell, a liver tumor cell, a breast tumor cell, or a brain tumor cell.

In another aspect, the invention features a method for identifying an agent that reduces cancer in a non-human animal. The method includes (a) administering a test agent to the animal, wherein the animal contains tumor cells originating, in the animal, from at least one heterologous stem cell, and (b) determining whether or not the test agent reduces the number of the tumor cells within the animal, wherein a reduction in the number of the tumor cells within the animal indicates that the test agent is the agent. The non-human animal can be a rodent (e.g., rat or mouse). The stem cell can contain a genetic alteration. The genetic alteration can contain an introduced nucleic acid molecule. The introduced nucleic acid molecule can encode a polypeptide. The polypeptide can be an oncogene product. The genetic alteration can contain a mutation. The mutation can be in a tumor suppressor gene. The tumor cells can be pancreatic tumor cells, liver tumor cells, a breast tumor cell, or brain tumor cells.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description.

### DESCRIPTION OF DRAWINGS

FIG. 1. Comparison of Ink4a/Arf+/+ and −/− neural stem cells (NSCs) and astrocytes. A. NSC morphology (upper panels) and nestin staining (inset upper panels) of neurospheres is similar for Ink4a/Arf+/+ and −/− cultures. Astrocyte morphology (lower panels) and GFAP staining (inset lower panels) is also similar between Ink4a/Arf+/+ and −/− cultures. B. The total number of EGF responsive NSCs isolated from Ink4a/Arf+/+ and −/− brains at E8.5 (n=4), E10.5 (n=9), E13.5 (n=38), E17.5 (n=12), P1 (n=16), and adult (6 weeks, n=4). C. The total number of neurospheres generated in defined media with EGF (20 ng/mL), without EGF, and with PDGF (50 ng/mL). Data represent the means +/− the standard error of the mean (SEM) of the number of stem cells residing in the striatal germinal zone at E13.5 (n=32-38 embryos per genotype). D. Differentiation of Ink4a/Arf−/− NSCs (nestin positive) into astrocytes (GFAP positive, lower left) in response to serum and neurons (TUJ1, lower right) in response to BDNF.

FIG. 2. p16^{INK4a} and p19^{ARF} cooperate to regulate the growth of astrocytes but not NSCs. Growth during serial passage by Ink4a/Arf genotype for A. NSCs and B. astrocytes. C. Number of persistently growing astrocytes lines (i.e., “non-senesced”; Sharpless et al., Nature, 413:86-91 (2001)) by passage and p16^{INK4a} and p19^{ARF} status. D. Western blot analysis of p16^{INK4a} and p19^{ARF} in NSCs and astrocytes by Ink4a/Arf genotype. +Control=p16^{INK4a} and p19^{ARF} overexpressing tumor cell line.

FIG. 3. Ink4a/Arf−/− astrocytes dedifferentiate to nestin+, A2B5+ progenitor cells in vitro. Ink4a/Arf+/+ (A) and −/− (B) cells were removed from serum and grown in EGF on day 0. Ink4a/Arf−/− cells rapidly change morphology and resulting bipolar cells and neurospheres are nestin+ and A2B5+ (double labeling inset, far right panel of B), whereas Ink4a/Arf+/+ cells do not dedifferentiate and remain GFAP+ (inset, far right panel of A). Western blot analysis of cultured astrocytes of indicated genotypes after treatment with EGF. C) Equivalent MAPK, AKT and D) EGFR phosphorylation is seen in Ink4a/Arf−/− and +/+ cells after EGF exposure. nestin and GFAP expression in the brains of adult Ink4a/Arf+/+ (A) and −/− (B) mice after intraventricular EGF infusion. Images (low and high power H&E, nestin and Olig2 staining) of E) Ink4a/Arf+/+ and F) −/− mice after intraventricular for 7 days of EGF. Arrows (3E) indicate a well-differentiated ependymal layer of single cell that is replaced by an expanded population of poorly differentiated progenitor cells (bracket, 3F).

FIG. 4. Expression of EGFR* in Ink4a/Arf−/− NSCs and astrocytes induces high-grade gliomas. Tumors derived from orthotopically transplanted Ink4a/Arf−/− EGFR* (A) NSCs and (B) astrocytes are gadolinium enhancing on MRI, grow as poorly differentiated high-grade tumors (40× H&E), and express GFAP, nestin, and olig2.

FIG. 5. A. p53−/−, p16^{INK4a}−/− and p19^{ARF}−/− astrocytes do not differentiate in response to EGF. Cultures were grown in serum-free media supplemented with EGF (20 ng/mL) for 10 days. In contrast to Ink4a/Arf−/− astrocytes, p53−/−, p16^{INK4a}−/−, and p19^{ARF}−/− astrocytes did not change morphology in response to EGF and remained GFAP+ and nestin− (insets represent double labeling with GFAP (red) and nestin (green) (n=4 independently derived cell lines for each genotype). B. Ink4a/Arf−/− astrocytes expressing the wild-type EGFR do not dedifferentiate in serum-free media containing without EGF. C. Ink4a/Arf−/− astrocytes expressing EGFR* dedifferentiate in serum-free media lacking EGF. D. Ink4a/Arf+/+ astrocytes expressing EGFR* do not dedifferentiate. E. EGFR* expression in NSCs can substitute for ligand. Ink4a/Arf−/− EGFR* NSC cultures were grown in serum free media without EGF. Ink4a/Arf−/− cultures transduced with the wild-type EGFR do not proliferate under these conditions, but rather undergo apoptosis (not shown). F. Subcutaneous tumors derived from Ink4a/Arf−/− astrocytes transduced with EGFR*. High grade, undifferentiated tumors were GFAP+, nestin+ and Olig2+, similar to intracranially generated tumors. Similar histology and immunoreactivity to GFAP, nestin, and Olig2 were found in subcutaneous tumors derived from Ink4a/Arf−/− NSCs transduced with EGFR* (not shown). G. Spindle-cell and epithelioid-cell morphology was seen in 1 tumor each derived from. All tumors demonstrated strong hEGFR* staining and were Sox10 positive as shown here for 1 tumor derived from Ink4a/Arf−/− EGFR* astrocytes.

### DETAILED DESCRIPTION

The invention provides methods and materials related to cancer models and the treatment of cancer. In particular, the invention provides chimeric non-human animals, methods for making and using chimeric non-human animals, isolated stem cells, and methods for identifying agents that reduce cancer in a non-human animal. For example, the invention relates to using stem cells to make chimeric non-human animals having cancer or the ability to develop cancer.

1. Chimeric Non-Human Animals

The invention provides non-human animals having heterologous stem cells. The term “non-human animal” refers to any animal other than a human. Examples of non-human animals include, without limitation, aquatic animals (e.g., fish, sharks, dolphin, and the like), farm animals (e.g., pigs, goats, sheep, cows, horses, rabbits, and the like), rodents (e.g., rats, guinea pigs, and mice), non-human primates (e.g., baboon, monkeys, and chimpanzees), and domestic animals (e.g., dogs and cats). The non-human animals provided herein can be immunocompromised or immunodeficient. For example, a non-human animal can be a SCID animal (e.g., an X-linked SCID or a RAG1−/− or RAG2−/− animal). The term “heterologous” as used herein with reference to a particular animal and a cell refers to any cell that is genetically different from the cells normally found in that particular animal. For example, all human cells are heterologous to a mouse. In addition, mouse cells extracted from a particular mouse, genetically altered (e.g., transfected with a construct that expresses a cDNA), and implanted back into that same particular mouse would be considered heterologous to that particular mouse provided the cells normally found in that particular mouse lack the introduced genetic alteration. Further, mouse cells extracted from one strain of mice are heterologous to a mouse of a different strain.

The term “stem cell” as used herein refers to an unspecialized cell that gives rise to a differentiated cell. For example, a NSC is an unspecialized neural cell that can give rise to a specialized neural cell such as a neuron, astrocyte, or oligodendrocyte. Stem cells can be identified from other cells using cell markers. Typically, pluripotent neural stem cells express nestin; multipotent glial progenitor cells express A2B5; neuronal progenitor cells express Pax6, MAP2, and TuJ1; oligodendrocyte progenitor cells express Olig2 and Sox10; immature oligodendrocytes express O4; mature oligodendrocytes express MBP and GalC; immature astrocytes express S100β; mature astrocytes express GFAP; mature neurons express NeuN and synaptophysin; liver stem cells express PDX-1, AFP, Cytokeratin19, Cytokeratin 14, and OV-6; mature hepatocytes express albumin, transferrin, and alpha-1-antitrypsin; pancreas stem cells express PDX-1 and Cytokeratin 19; mature exocrine pancreatic cells express carboxypeptidase-A and amylase; and mature endocrine pancreatic cells express glucagons and insulin.

The non-human animals provided herein can contain any type of stem cell. For example, a non-human animal can be made to contain embryonic stem cells or tissue-specific stem cells. Examples of tissue-specific stem cells include, without limitation, NSCs, liver-specific stem cells, pancreas-specific stem cells, hematopoietic-specific stem cells, mammary-specific stem cells, and bone marrow stromal-specific stem cells. In addition, stem cells can be from any type of animal including, without limitation, humans, monkeys, pigs, dogs, rabbits, guinea pigs, mice, and rats. Any method can be used to obtain stem cells. Such methods include, without limitation, standard stem cell isolation techniques and cell sorting techniques (See, e.g., Gussoni et al., Nature 401:390-394 (1999); Brustle et al., Science, 285:754-756 (1999); and Krause et al., Cell, 105:369-377 (2001)). Alternatively, stem cell lines can be obtained from various public and private sources such as tissue depositories.

The stem cells within a non-human animal can be dedifferentiated cells. For example, a stem cell can be a cell that was dedifferentiated from a specialized cell (e.g., an astrocyte) to form an unspecialized cell (e.g., a NSC). Any specialized cell can be dedifferentiated to form a stem cell. For example, astrocytes, pancreatic acinar cells, melanocytes, and hepatocytes can be dedifferentiated to form stem cells. Typically, dedifferentiated cells are produced from a cultured specialized cell (e.g., cultured astrocytes, cultured pancreatic acinar cells, cultured melanocytes, and cultured hepatocytes). Any method can be used to obtain dedifferentiated cells. Such methods include, without limitation, the genetic methods and culturing methods described herein.

Any method can be used to make non-human animals containing heterologous stem cells. For example, stem cells can be injected into a non-human animal. The stem cells can be implanted such that the recipient tissue is tissue in an adult non-human animal. While stem cells can be administered to any site including blood, they typically are administered to a particular tissue (e.g., bone, muscle, lung, pancreas, prostate, liver, or brain). For example, NSCs can be directly injected into brain tissue, while liver-specific stem cells can be injected into liver tissue or the portal vein. Other routes of administration include, without limitation, intravenous, intraperitoneal, intramuscular, subcutaneous, intrathecal, and intradermal administrations. The stem cells can be administered via a single administration or multiple administrations (e.g., two, three, four, or more administrations). In addition, any amount of stem cells can be administered to a non-human animal to make a non-human animal containing heterologous stem cells. Typically, the number of stem cells administered to rodents is between about 10² and about 10¹⁰ stem cells (e.g., about 10³, 10⁴, 10⁵, 10⁶, 10⁷, 10⁸, 10⁹ stem cells per administration). More than 10¹⁰ stem cells can be used when making non-human animals larger than rodents.

The non-human animals provided herein can contain stem cells having a genetic alteration. For example, the stem cells within a non-human animal can be designed to contain an introduced nucleic acid molecule. Typically, the introduced nucleic acid molecule is incorporated into the genome of the stem cell. Any method can be used to introduce a nucleic acid molecule into a stem cell. For example, calcium phosphate precipitation, electroporation, lipofection, microinjection, and viral-mediated nucleic acid transfer methods can be used to introduce nucleic acid molecules into stem cells. In addition, nucleic acid molecules can be introduced into stem cells using transgenic technology.

In addition, the introduced nucleic acid molecule can encode a polypeptide. Such polypeptides include, without limitation, transcription factors, enzymes (e.g., telomerase), receptors, ligands, adhesion molecules, transporters, oncogene products, and tumor suppressor gene products. For example, the encoded polypeptide can be a polypeptide involved in cell cycle control, cell survival, cell invasion, or metastasis. In some embodiments, human stem cells can be engineered to contain a nucleic acid molecule that encodes a polypeptide having telomerase activity. The term “oncogene product” as used herein refers to a polypeptide encoded by an oncogene. The term “tumor suppressor gene product” as used herein refers to a polypeptide encoded by a tumor suppressor gene. Examples of oncogene products include, without limitation, K-RAS, H-RAS, epidermal growth factor receptor (EGFR), MDM2, HER2/Neu, erb-B2, TGFβ, RhoC, AKT family members, myc, cyclin D1, prolactin, β-catenin, PGDF, C-MET, PI3K-CA, CDK4, and Bcl2 anti-apoptotic family members (e.g., Bcl2) as well as their activated forms. The activated form of EGFR is designated EGFR* herein. Examples of tumor suppressor gene products include, without limitation, p16^{INK4a}, p19^{ARF} (p14^{ARF} for humans), p53, PTEN, pRB, SMAD4, MAD family members (e.g., MXI1), APC(MIN), LKB1, LATS, Apaf1, Caspase 8, APC, DPC4, KLF6, GSTP 1, ELAC2/HPC2 or NKX3.1, ATM, CHK2, ATR, BRCA1, BRCA2, MSH2, MSH6, PMS2, Ku70, Ku80, DNA/PK, XRCC4, and MLH1. Other examples of tumor suppressor gene products include, without limitation, those polypeptides involved in the p53 and/or pRB pathways. The introduced nucleic acid molecule can encode antisense molecules and/or ribozymes.

The genetic alteration to the stem cell can include a mutation. For example, the genomic sequence of a stem cell can be mutated such that a mutated version of a polypeptide is expressed. In this case, the mutated polypeptide can have a function that is different from the function of the unmutated polypeptide. Alternatively, the genomic sequence of a stem cell can be mutated such that the expression of a polypeptide is increased or decreased. Polypeptide expression can be increased by inserting a strong promoter sequence upstream of the polypeptide-encoding sequence. Polypeptide expression can be reduced by disrupting the nucleic acid sequence that encodes the polypeptide (e.g., a tumor suppressor gene product). Techniques similar to those used to make knock-out mice can be used to disrupt nucleic acid sequences. Other techniques that can be used to reduce polypeptide expression include, without limitation, antisense technology, RNA interference techniques, and ribozyme technology. RNA interference can be induced by introducing double-stranded RNA complementary to a target messenger RNA into cells. The target messenger RNA can be transcribed from a tumor suppressor gene such as p53, p16, p27, PTEN, BRCA1, and BRCA2. Genetic alteration can be introduced into a stem cell using chemical mutagenesis techniques as well.

Any nucleic acid sequence can be genetically altered. For example, intronic sequences, exonic sequences, and regulatory sequences (e.g., promoters, enhancers, and silencers) can be altered. In addition, tumor suppressor genes such as INK4a/ARF, p53, and PTEN can be altered. For example, a tumor suppressor gene of a stem cell can be genetically altered such that the stem cell exhibits reduced tumor suppressor activity. Reductions in tumor suppressor activity can be determined by examining the cells for the expression of specific tumor suppressor gene products. For example, western blots can be used to determine whether or not a cell population expresses a particular tumor suppressor gene product. In addition, reductions in tumor suppressor activity can be assessed functionally by inserting the cells into an animal and monitoring those cells for the ability to form tumor cells. Cells having reduced tumor suppressor activity can form tumors more frequently and/or more quickly than cells not having reduced tumor suppressor activity.

In addition, the stem cells can be genetically altered to contain nucleic acid sequences that are regulated in an inducible manner. For example, an introduced nucleic acid molecule can be designed to encode an oncogene product under the control of an inducible promoter system such as the tetracycline-regulated promoter system described elsewhere (See, e.g., PCT/US02/09710). In this case, administering the inducing agent (e.g., tetracycline or doxycycline) to the animal via, for example, the animal's drinking water can result in expression of the encoded polypeptide. Further, introduced nucleic acid sequences can contain polypeptide-encoding sequences operably linked to a promoter sequence. The promoter sequence can be a general promoter (e.g., the cytomegalovirus (CMV) promoter) or a tissue-specific promoter (e.g., a tyrosinase promoter to express a polypeptide in a melanoma cell; a TRP2 promoter to express a polypeptide in a melanocytes; an MMTV or WAP promoter to express a polypeptide in breast cells and/or cancers; a Villin or FABP promoter to express a polypeptide in intestinal cells and/or cancers; a RIP promoter to express a polypeptide in pancreatic beta cells; a Keratin promoter to express a polypeptide in keratinocytes; a Probasin promoter to express a polypeptide in prostatic epithelium; a nestin or GFAP promoter to express a polypeptide in CNS cells and/or cancers; a tyrosine hydroxylase or S100 promoter to express a polypeptide in neurons; and an Alpha myosin promoter to express a polypeptide in cardiac cells). For example, embryonic stem cells can be designed to contain a tyrosine hydroxylase promoter sequence operably linked to a nucleic acid sequence that encodes an oncogene such that cells that differentiate into, for example, dopaminergic neurons can express the encoded oncogene product.

The stem cells can be genetically altered to contain nucleic acid sequences that can excise a nucleic acid sequence in a regulated manner. For example, cre-lox systems can be used to excise nucleic acid flanked by LoxP sites. In one example, stem cells can be designed to contain a tumor suppressor gene flanked by LoxP sites. In this case, induction of cre recombinase expression can result in the removal of the tumor suppressor gene sequences flanked by the LoxP sites, thus reducing expression of the tumor suppressor gene product.

In one embodiment, the heterologous stem cells of a non-human animal are genetically altered such that they are tumorigenic. For example, stem cells can be genetically altered such that they are more tumorigenic than stem cells lacking the genetic alteration. Typically, tumorigenic stem cells can be made by inserting one or more nucleic acid molecules that encode oncogene products and/or by reducing the expression of one or more tumor suppressor gene products. Examples of genetic alterations that can be used to produce tumorigenic stem cells include, without limitation, genetic alterations that result in EGFR* expression in combination with reduced p16^{INK4a} and reduced p19^{ARF} expression (e.g., genetic alterations that produce an EGFR*⁺ and INK4a/ARF^{-/-} genotype), genetic alterations that result in PDGF expression in combination with reduced p53 expression (e.g., genetic alterations that produce an PDGF⁺ and p53^{-/-} genotype), genetic alterations that result in TGFα expression in combination with reduced p53 expression (e.g., genetic alterations that produce an TGFα⁺ and p53^{-/-} genotype), and genetic alterations that result in reduced PTEN, p16^{INK4a}, and p19^{ARF} expression (e.g., genetic alterations that produce an PTEN^{-/-} and INK4a/ARF^{-/-} genotype).

Stem cells having genetic alterations that result in (1) EGFR* expression in combination with reduced p16INK4a and reduced p19ARF expression or (2) PDGF expression in combination with reduced p53 expression can develop into brain tumor cells. Stem cells having genetic alterations that result in TGFα expression in combination with reduced p53 expression can develop into pancreatic tumor cells. Stem cells having genetic alterations that result in reduced PTEN, p16^{INK4a}, and p19^{ARF} expression can develop into lymphomas, gastrointestinal lymphomas, pseocromocytomas, and sarcomas.

The genetic alterations can be performed in vitro. For example, stem cells in culture can be genetically altered by infecting the cells with a viral vector (e.g., retroviral vectors such as murine leukemia viral vectors) having the ability to integrated into the genome of infected cells. Other viral vectors that can be used to introduce nucleic acid into stem cells include, without limitation, adenovirus vectors, herpes virus vectors, and lentiviral vectors. Alternatively, genetic alterations can be introduced into stem cells via standard transgenic and/or knock-out techniques. For example, stem cells expressing EGFR* can be obtained from EGFR*⁺ animals produced using standard transgenic technology. Likewise, stem cells having an INK4a/ARF^{-/-} genotype can be obtained from INK4a/ARF^{-/-} animals produced using standard knock-out technology.

The tumorigenic stem cells can be highly tumorigenic, moderately tumorigenic, or slightly tumorigenic. The term “highly tumorigenic” as used herein with reference to cells refers to cells that, in a standard tumorigenic assay, result in (1) at least sixty percent of test animals developing tumor cells within twelve weeks of administration, (2) at least eighty percent of test animals developing tumor cells within twenty weeks of administration, or (3) at least ninety percent of test animals developing tumor cells within six months of administration. The term “moderately tumorigenic” as used herein with reference to cells refers to cells that, in a standard tumorigenic assay, result in (1) between thirty and fifty-nine percent of test animals developing tumor cells within twelve weeks of administration, (2) between thirty and seventy-nine percent of test animals developing tumor cells within twenty weeks of administration, or (3) between forty and eighty-nine percent of test animals developing tumor cells within six months of administration. The term “slightly tumorigenic” as used herein with reference to cells refers to cells that, in a standard tumorigenic assay, result in (1) between five and twenty-nine percent of test animals developing tumor cells within twelve weeks of administration, (2) between fifteen and twenty-nine percent of test animals developing tumor cells within twenty weeks of administration, or (3) between fifteen and thirty-nine percent of test animals developing tumor cells within six months of administration.

The standard tumorigenic assay is performed by administering 10⁵ cells/animal on day zero via a single injection. The concentration of each administration is between 10³ to 10⁴ cells/μL. The cell are injected into the appropriate tissue or compartment depending on the type of cell. For example, NSCs are injection into brain tissue, while liver-specific stem cells are injected into the portal vein, and hematopoietic-specific stem cells are injected into the blood. Embryonic stem cells are injecting into any region of the animal. For example, embryonic stem cells can be injected into a neural compartment (e.g., brain) or mammary tissue. For each time point to be tested (e.g., twelve weeks, twenty weeks, and six months following administration), at least ten animals are used. Once the time point to be tested is reached, the animals for that group are examined for the presence of tumor cells. Any method can be used to identify the presence of tumor cells. For example, tissue samples can be examined histologically, blood samples can be evaluated for the presence of cancer markers, or the health of the animal can be assessed for the presence of clinical signs of cancer. Other methods that can be used to identify animals containing tumor cells include, without limitation, those methods routinely used to identify tumor cells in humans (e.g., ultrasound technology, x-rays, cell-based staining assays, PCR-based assays, and biochemical assays). In addition, animal survival can be used to identify animals containing tumor cells. In this case, the presence of tumor cells can be confirmed by examining the animal's body for signs of cancer.

The invention also provides non-human animals containing tumor cells that originate in the animals from at least one heterologous stem cell. Again, the heterologous stem cells can be any of the stem cells described herein. For example, the stem cells can be tissue-specific stem cells (e.g., NSCs) having a genetic alteration (e.g., an introduced nucleic acid molecule that encodes EGFR*). A tumor cell within a non-human animal can be any type of tumor cell such as a pancreatic tumor cell, liver tumor cell, primary brain tumor cell (e.g., glioma), lymphoma cell (e.g., acute lymphoblastic leukemia cell), sarcoma cell, non-small cell lung carcinoma cell, renal cell carcinoma cell, head and neck tumor cell, prostate tumor cell, bladder carcinoma cell, or basal cell carcinoma cell. In addition, a tumor cell within a non-human animal originates from an implanted stem cell when that tumor cell arises from the implanted stem cell or any cell (e.g., a differentiated cell) derived from the implanted stem cell. The relationship between tumor cells and implanted stem cells can be confirmed using various techniques including, without limitation, immunological assays, biochemical assays, and genetic assays. For example, PCR can be used to identify tumor cells having the same genetic markers as those possessed by the implanted stem cells. For the purposes of this invention, tumor cells are said to originate or develop from implanted stem cells when genetic or biochemical tests reveal that the tumor cells are more like the implanted stem cells than the cells of the recipient animal. For example, when human stem cells are administered to a mouse and tumor cells develop, those tumor cells will be considered cells originating from the human stem cells provided those tumor cells genetically resemble human cells as opposed to mouse cells. It is noted that stem cells can be designed to express a detectable polypeptide marker (e.g., GFP or luciferase) not expressed by the recipient animal. In such a case, cells expressing the detectable polypeptide marker can be identified as originating or developing from the implanted stem cell as opposed to the animal's cells.

2. Isolated Stem Cells

The invention provides isolated stem cells. Such stem cells can contain a genetic alteration that makes the stem cells tumorigenic (e.g., highly, moderately, or slightly tumorigenic). For example, the isolated stem cells can contain an introduced nucleic acid molecule that encodes an oncogene product. The isolated stem cells can be INK4a/ARF^{-/-} stem cells. Typically, INK4a/ARF^{-/-} stem cells are obtained from an INK4a/ARF^{-/-} animal (e.g., an INK4a/ARF^{-/-} knock-out mouse). When isolating INK4a/ARF^{-/-} stem cells from an animal, any tissue can be used. For example, INK4a/ARF^{-/-} stem cells can be isolated from brain tissue, pancreas tissue, or liver tissue. The isolated stem cells can be dedifferentiated cells as described herein. For example, isolated stem cells can be dedifferentiated astrocytes maintained in culture. The methods described herein can be used to obtain isolated dedifferentiated cells.

Any method can be used to identify stem cells. Such methods include, without limitation, cell staining techniques that label polypeptides associated with undifferentiated stem cells.

3. Identifying Agents that Reduce Cancer in Non-Human Animals

Agents that reduce cancer in a non-human animal can be identified by (1) administering a test agent to a non-human animal containing tumor cells, and (2) determining whether or not that administered test agent reduces the number of tumor cells within the animal, reduces tumor migration, reduces angiogenesis, and/or prevents an increase in the number of tumor cells. Any of the non-human animals provided herein can be used to identify agents that reduce cancer in a non-human animal. Test agents can be any type of molecule having any chemical structure. For example, a test agent can be a polypeptide (e.g., an antibody), carbohydrate, small molecule compound, lipid, amino acid, ester, alcohol, carboxylic acid, nucleic acid, fatty acid, or steroid. In addition, test agents can be lipophilic, hydrophilic, hydrophobic, plasma membrane permeable, or plasma membrane impermeable.

The test agents can be administered to the non-human animal via any route. For example, the test agent can be administered systemically, intravenously, intraperitoneally, intramuscularly, subcutaneously, intrathecally, intradermally, or orally. In addition, any amount of the test agent can be administered. Typically, a test agent is administered to the non-human animal in an amount large enough to ensure significant delivery of the agent to the tumor cells without causing significant adverse side effects to the animal. Standard pharmacological studies can be performed to assess the amount of agent being delivered to the tumor cells for a given amount administered. Based on these studies, the amount administered can be increased or decrease.

Any method can be used to determine whether or not the administered test agent had an anti-cancer effect (e.g., reduction in the number of tumor cells within the animal). For solid tumors, the diameter of the tumor can be measured before and after administration. Such measurements can be made using a caliper when the tumor has a dermal location. When the tumor occurs in a visceral cavity (e.g., liver or lung) or intracranially (e.g., within the brain), imaging techniques such as contrast enhanced computed tomography (CT) or magnetic resonance imaging (MRI) can be used to measure the size of tumors. Reductions in other types of tumor cells can be assessed using histological, biochemical, immunological, or clinical techniques. For example, histological techniques can be used to determine whether or not tumor cells remain in a particular tissue. Likewise, clinical techniques can be used determine the health of an animal thereby assessing the stage of cancer progression. If cancer progression stops or is reversed, then the number of tumor cells within the animal was, most likely, reduced. Additional studies can be used to confirm an anti-caner effect such as flow assisted cell sorting (FACS) or in vivo fluorescent imaging from cell genetically altered to express, for example, luciferase activity.

The invention will be further described in the following examples, which do not limit the scope of the invention.

### EXAMPLES

### Example 1

Astrocyte Dedifferentiation and Production of Chimeric Cancer Models

Loss of Ink4a/Arf (p16^{INK4a} and p19^{ARF}) tumor suppressor function and activation of the epidermal growth factor receptor (EGFR) are signature changes encountered in the high-grade malignant gliomas. The combined loss of p16^{INK4a} and p19^{ARF}, but not loss of p53, p16^{INK4a}, or p19^{ARF} alone, enables astrocyte dedifferentiation in response to EGFR pathway activation. Dedifferentiated astrocytes acquire morphological and functional properties of neural stem cells (NSCs) and/or early glial progenitors in vitro. Moreover, when Ink4a/Arf−/− NSCs and astrocytes are transduced with a constitutively active EGFR mutant allele, both cell types generate poorly differentiated high-grade gliomas with overlapping clinicopathological features. These findings identify both NSCs and astrocytes as equally permissive compartments for gliomagenesis and provide evidence for a synergistic role for p16^{INK4a} and p19^{ARF} in the maintenance of terminal astrocyte differentiation. Thus, dysregulation of specific genetic pathways, rather than the cell-of-origin, appears to dictate the emergence and phenotype of high-grade gliomas.

This work, demonstrating that both astrocytes and neural stem cells can serve as the cell-of-origin for high-grade malignant gliomas, addresses a longstanding debate in the field of neuro-oncology and neurodevelopment. The central finding is that a combination of EGF pathway activation and loss of both p16^{INK4a} and p19^{ARF} tumor suppressor function provokes a common high-grade glioma phenotype regardless of the specific cell-of-origin. Moreover, this work identifies a new function for Ink4a/Arf tumor suppressor, and interaction with EGFR activation, in the maintenance of astrocyte terminal differentiation. Together, these findings provide insight into the cellular origins of malignant glioma and the role of its signature genetic lesions in pathogenesis of these highly lethal brain cancers.

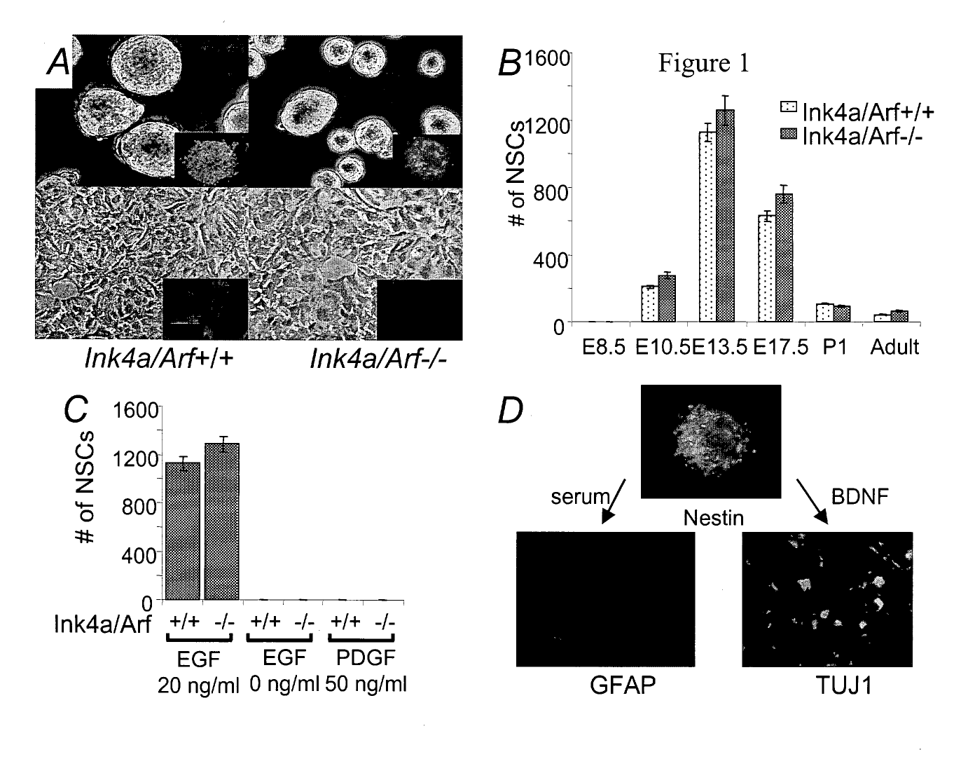

The dispensability of p16^{INK4a} and p19^{ARF} function in brain development was confirmed by an extensive histological and morphometric analysis of the developing brain from Ink4a/Arf−/− mutants and control littermates on embryonic days E13.5, 15.5, and 19, postnatal days 1 and 25, and aging adults (data not shown). These studies were complemented by a series of cell culture-based analyses of the growth and differentiation of NSCs and cortical astrocytes. The various Ink4a/Arf genotypes exhibited similar morphology and differentiation markers of primary NSC and astrocyte cultures (FIG. 1A) and showed comparable emergence of an EGF-responsive NSC pool on E10.5, with expansion through E13.5 (FIG. 1B)—kinetics that reflect the increased EGF responsiveness during development (Chiasson et al., J. Neuroscience, 19:4462-4471 (1999) and Qian et al., Neuron 28:69-80 (2000)). Similar to littermate wildtype cultures, Ink4a/Arf−/− NSCs retained strict EGF-dependence for growth, and PDGF substitution was unable to sustain wildtype or mutant cultures (FIG. 1C). Moreover, Ink4a/Arf status did not affect the capacity of NSCs to differentiate into GFAP-positive astrocytes (93+/−4%) and TUJ1-positive neurons (40+/−5%) in response to serum and BDNF, respectively (FIG. 1D).

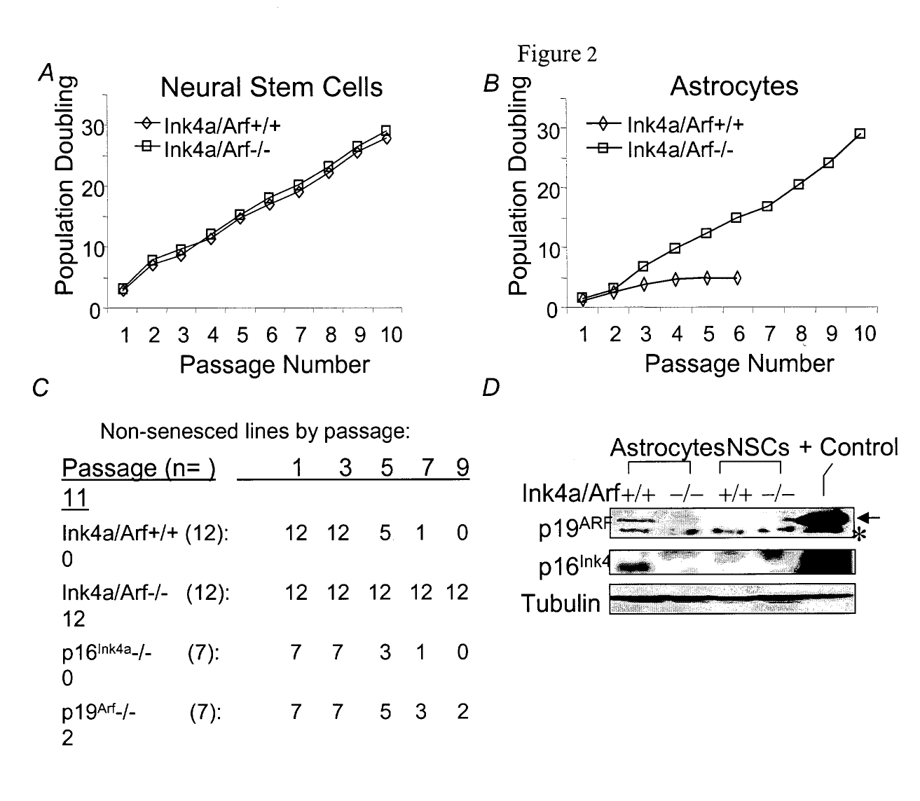

A detailed proliferation analysis of early passage NSC and astrocyte cultures was conducted. Although Ink4a/Arf+/+ and −/− NSCs (FIG. 2A) exhibited comparable growth rates, there was a striking increase in the proliferative rate of Ink4a/Arf−/− astrocytes relative to wildtype cultures (FIG. 2B). Correspondingly, cell cycle analysis of early passage astrocyte cultures showed marked differences in the S phase fraction (33% for Ink4a/Arf−/− versus 12% for +/+ astrocytes, n=5 cultures each, p<0.005). As shown previously for oligodendrocyte precursors (Tang et al., Science 291:868-871 (2001)), Ink4a/Arf+/+ NSCs proliferated indefinitely if prevented from differentiating. In contrast, serially passaged Ink4a/Arf−/− astrocytes exhibited immortal growth while Ink4a/Arf+/+ astrocytes, as well as those specifically deficient for p16^{INK4a} (Sharpless et al., Nature, 413:86-91 (2001)) or p19^{ARF} underwent proliferative arrest and assumed senescent features by 5 to 7 population doublings (FIG. 2C). p19^{ARF}−/−, but not p16^{INK4a}−/− or Ink4a/Arf+/+ cultures, exhibited a modest rate of escape from senescence (29%, FIG. 2C), while 100% of Ink4a/Arf−/− cultures were immortal. Consistent with these observed developmental stage-specific differences in growth, p16^{INK4a} and p19^{ARF} were undetectable in extensively passaged wild-type NSC cultures, whereas both p16^{INK4a} and p19^{ARF} were readily detected in cultured Ink4a/Arf+/+ astrocytes (FIG. 2D). Taken together, these data demonstrate that both p16^{INK4a} and p19^{ARF} play an important, yet developmentally restricted, role in the control of glial lineage proliferation in vitro.

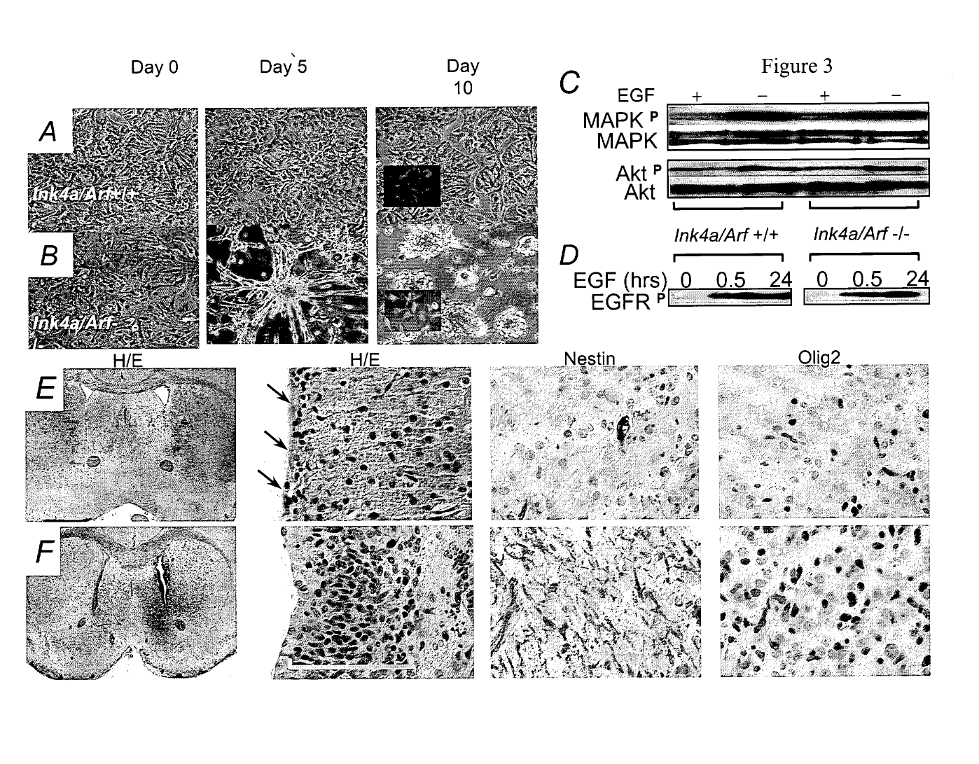

To investigate the transition to a poorly differentiated glial phenotype in progression from low-grade astrocytoma to secondary GBM, the roles of Ink4a/Arf and the EGFR pathway in the maintenance of astrocyte differentiation were assessed. Subconfluent cycling early passage GFAP+ primary astrocytes were removed from serum-supplemented media and exposed to serum-free media containing EGF (20 ng/mL). Under these conditions, all Ink4a/Arf+/+ cultures as well as astrocyte cultures derived from p16^{INK4a} null and p19^{ARF} null mice, survived, continued to proliferate, and retained the morphological and immunohistochemical features of fully differentiated astrocytes (FIG. 3A). In sharp contrast, EGF-treated Ink4a/Arf−/− astrocytes showed contracted cytoplasm within 24 hours and, over the course of 7 to 10 days, developed into bipolar cells coexisting with small neurospheres which detached from the culture dish and continued to proliferate as substrate-independent neurospheres (FIG. 3B)—properties ascribed to NSCs. While littermate wildtype astrocyte cultures retained GFAP expression in EGF (FIG. 3B, inset), Ink4a/Arf−/− cultures demonstrated complete loss of GFAP and induction of the progenitor markers nestin and A2B5 (FIG. 3B inset). These Ink4a/Arf-dependent responses were not related to differences in EGFR expression or activation, as evidenced by a comparable degree of EGF-induced EGFR phosphorylation (FIG. 3D) and activation of the EGFR signaling surrogates, MAPK and AKT (FIG. 3C). Together, these data indicate that EGFR pathway activation can recruit fully differentiated astrocytes to dedifferentiate into a progenitor phenotype, and that this process is restrained by the combined actions of p16^{INK4a} and p19^{ARF}.

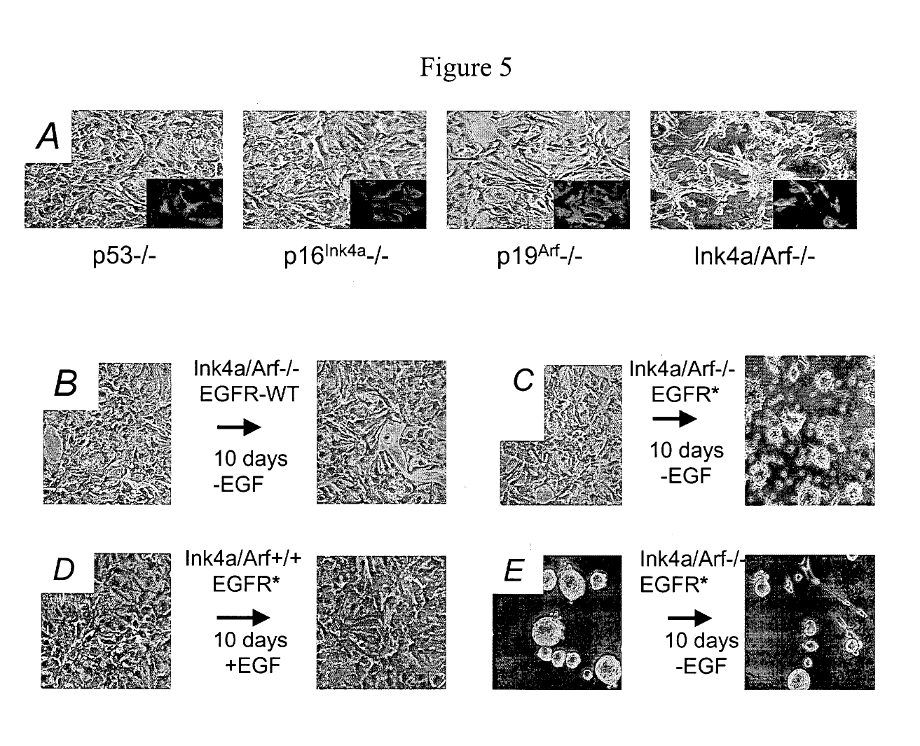

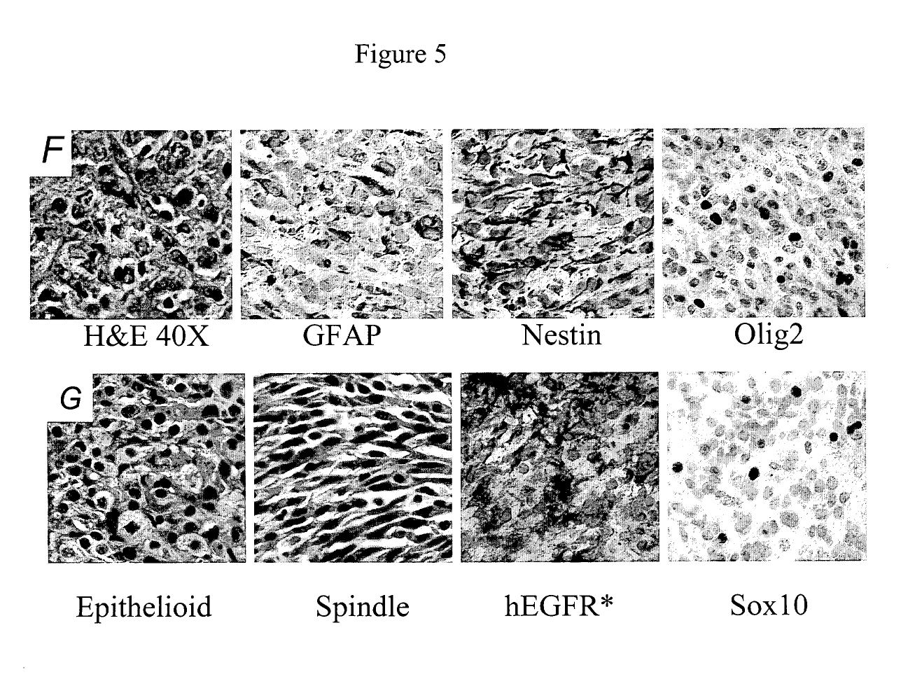

To understand further these synergistic actions, we asked whether immortalization per se or the capacity for rapid proliferation represent the key parameters that endow EGF-induced astrocyte plasticity. Despite rapid rates of growth and an immortal phenotype, p53−/− astrocyte cultures subjected to prolonged EGF treatment failed to induce morphological or immunohistochemical changes consistent with dedifferentiation (FIG. 5A). Likewise, astrocytes from mice singly deficient for either p16^{INK4a} or p19^{ARF} did not dedifferentiate in response to EGF (FIG. 5A). Furthermore, in contrast to a previous report showing PDGF induction of glial progenitor morphology and expression markers in wildtype astrocytes after extended passage in culture (40 days; Dai et al., Genes Dev., 15:1913-1925 (2001)), the placement of early-passage Ink4a/Arf−/−, p53−/− or Ink4a/Arf+/+ astrocyte cultures in serum-free media containing 50 ng/mL PDGF did not induce dedifferentiation (data not shown). Thus, the data provided herein provide a rational explanation for the observation of poorly differentiated histology, activated EGFR, and loss of Ink4a/Arf function in human high grade gliomas (Ekstrand et al., Cancer Res., 51:2164-2172 (1991)), and the correlation of aberrant PDGF signaling and loss of p53 function in low grade astrocytomas which have a more differentiated histologic phenotype (Kleihues and Cavenee, World Health Organization Classification of Tumours of the Nervous System, WHO.IARC (2000)). Moreover, these data establish the specificity and cooperativity of the EGFR pathway and Ink4a/Arf function in processes of astrocyte dedifferentiation.

To provide in vivo confirmation of the functional relationship between EGFR activation and the status of Ink4a/Arf, Ink4a/Arf +/+ and −/− adult mice underwent intraventricular infusion of EGF for 7 days. EGF treatment induced proliferation and diffuse infiltration of a population of poorly differentiated, nestin- and Olig2- (Lu et al., Proc. Natl. Acad. Sci. USA, 98:10851-10856 (2001) and Marie et al., Lancet, 358:298-300 (2001) positive cells, a response that was markedly enhanced in the setting of Ink4a/Arf deficiency (FIG. 3F and comparison with Ink4a/Arf+/+ FIG. 3E). Consistent with EGF-induced dedifferentiation of terminally differentiated cells was the complete replacement of the well-differentiated ependymal layer (arrows, FIG. 3E) by the poorly differentiated cells (bracket, FIG. 3F) in Ink4a/Arf−/−, but not Ink4a/Arf+/+, mice.

To determine whether cell-autonomous effects of this glioma-relevant lesion could induce astrocyte dedifferentiation in vitro, Ink4a/Arf+/+ and −/− astrocytes were inoculated with retrovirus encoding EGFR* or wild-type EGFR receptor (EGFR-WT) in serum-free media with EGF supplementation. Comparable expression of transduced EGFR* and EGFR-WT protein was documented by western blot analysis (not shown). Both EGFR* and EGFR-WT Ink4a/Arf−/− astrocytes recapitulated the aforementioned dedifferentiation response (n=4 cultures each), although this response required EGF supplementation in the case of EGFR-WT Ink4a/Arf−/− cultures (FIGS. 5B and 5C). EGF- and EGFR* induced dedifferentiation responses were similar except that the kinetics of dedifferentiation was faster in response to EGF (average of 8.5 versus 18 days, n=6 cultures each)—a finding consistent with a more modest activation of MAPK activation by EGFR*. Of note, despite robust and comparable levels of transduced EGFR* or EGFR-WT and activation of MAPK in EGF, Ink4a/Arf+/+ cultures did not dedifferentiate under any conditions (FIG. 5D, n=4 cultures each). That EGFR* was functionally activated was documented further by the survival of dedifferentiated Ink4a/Arf−/− EGFR* cultures and Ink4a/Arf−/− EGFR* NSCs in the absence of EGF supplementation (FIG. 5E). The findings corroborate the EGF ligand studies and indicate that these key glioma lesions drive the classical poorly differentiated glial phenotype of these cancers.

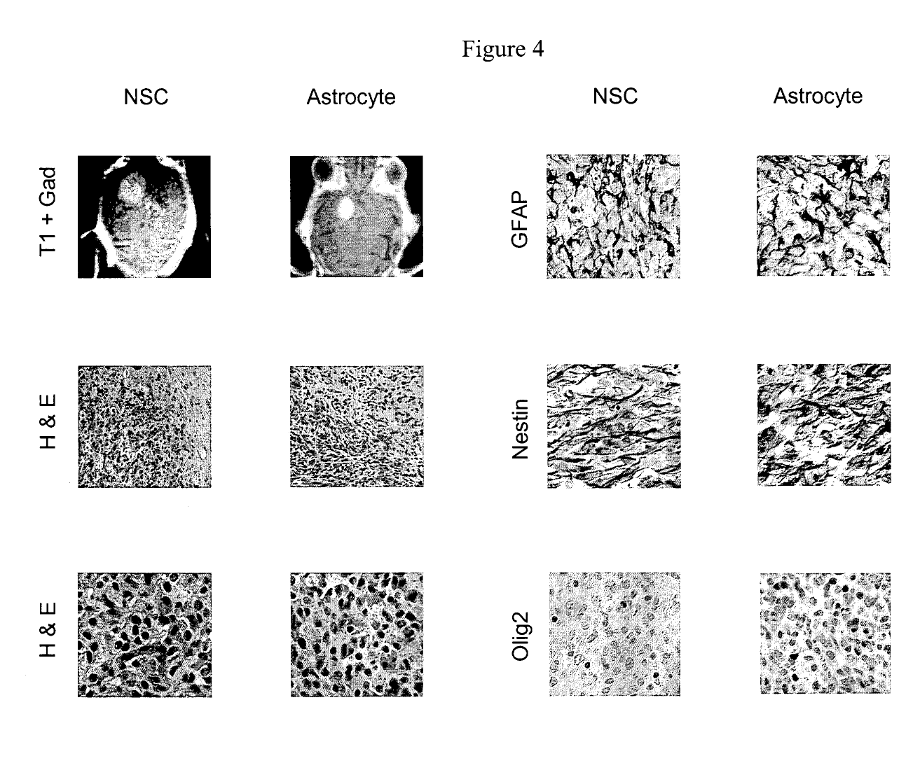

The genetically defined primary cultures described in this work provided an opportunity to assess directly how the state of glial differentiation influences the transforming actions of EGFR* in vivo. To this end, early passage EGFR* or EGFR-WT transduced NSCs (2×10⁴ cells per injection, n=6 per genotype) and astrocytes (2×10⁴ cells per injection, n=4 per genotype) from Ink4a/Arf−/− and +/+ mice were transplanted orthotopically into the brains of adult SCID mice and followed for 12 weeks or until development of focal neurological deficits (Table 1). Astrocyte cultures were maintained in serum prior to injection to avoid the possibility that tumor formation was a result of in vitro dedifferentiation. Neither Ink4a/Arf+/+ NSCs nor astrocytes yielded tumors or neurological deficits after 3 months, even when engineered to express EGFR* (or EGFR-WT) (Table 1). In contrast, EGFR* Ink4a/Arf−/− NSC and astrocyte cultures readily formed tumors with a latency of 4-8 weeks (FIG. 4). Clonality of these tumors was confirmed by the emergence of a distinct banding pattern on Southern blots assayed for hybridization to a Moloney LTR fragment when compared to parental cell lines (data not shown). The tumorigenic competence of EGFR* Ink4a/Arf−/− NSCs and astrocytes, as opposed to EGFR* Ink4a/Arf+/+ controls, was documented further by tumor formation following subcutaneous injections into SCID mice (FIG. 5F).

TABLE I
Growth of orthotopically transplanted astrocytes and
NSCs in SCID brains.

Transduced With:

| | EGFR WT | EGFR* |
|---|---|---|
| Ink4a/Arf+/+ Astrocytes | 0/4 | 0/4 |
| Ink4a/Arf−/− Astrocytes | 0/4 | 4/4 |
| Ink4a/Arf+/+ NSCs | 0/6 | 0/6 |
| Ink4a/Arf−/− NSCs | 0/6 | 6/6 |

Notably, despite being at opposite ends of the differentiation spectrum, both EGFR* Ink4a/Arf−/− NSCs (FIG. 4A) and astrocytes (FIG. 4B) yielded neoplasms with overlapping clinical, radiographic, and histopathological features. Clinically, all animals presented with focal neurological signs that included asymmetric hind and forelimb dystonia followed by progressive paraplegia. These symptoms matched precisely MRI findings of gadolinium enhancing mass lesions in the striatum and did not reflect neurological trauma associated with the injection (FIG. 4). Histologically, the tumors from both EGFR* Ink4a/Arf−/− NSCs and astrocytes resembled high-grade gliomas demonstrating hypercellularity, pleomorphism, high mitotic activity and focal invasion into normal parenchyma (FIG. 4). Five tumors derived from NSCs and 3 tumors derived from astrocytes were composed of small, undifferentiated cells (FIG. 4) while 1 tumor derived from NSCs was composed of spindle-shaped cells and 1 tumor derived from astrocytes was composed of epithelioid cells (FIG. 5G). This range of cellular morphologies is consistent with the ‘multiforme’ nature of human high-grade gliomas. Tumors derived from both NSCs and astrocytes were GFAP positive (FIG. 4) and strongly expressed hEGFR (FIG. 5G). Notably, as demonstrated for the proliferating population of cells in response to intraventricular infusion of EGF in Ink4a/Arf−/− mice (FIG. 3F), the tumor cells derived from both Ink4a/Arf−/− NSCs and astrocytes were strongly nestin- and Olig2-positive (FIG. 4), suggesting that the Ink4a/Arf−/− astrocytes transduced with EGFR* underwent dedifferentiation during transformation in vivo. That these cells did not represent normal oligodendrocytes intermingled with tumor was shown by the presence of Olig2+ cells in the subcutaneous tumor (FIG. 5F).

These results demonstrate that both NSCs and astrocytes can serve equally as the glioma cell-of-origin in the face of Ink4a/Arf deficiency and constitutive EGFR activation. That the tumors derived from both cell types are poorly differentiated and morphologically similar indicates that mature astrocytes undergo dedifferentiation during processes of transformation. Since neither p16^{INK4a} nor p19^{ARF} loss alone induced astrocyte immortalization or dedifferentiation in response to EGF, these results demonstrate a role for both proteins encoded by the Ink4a/Arf locus in gliomagenesis.

The increase in astrocyte proliferation in response to loss of both proteins is not sufficient for dedifferentiation since neither Ink4a/Arf−/− astrocytes in serum nor p53−/− astrocytes in EGF dedifferentiate. Although there is no evidence that p16^{INK4a} and p14^{ARF} are involved in maintaining astrocytes in a fully differentiated state in the normal human brain, the data suggests that they become important in regulating the response to oncogenic stimuli, such as EGFR activation. These findings gain added significance through the in vivo studies showing that intraventricular EGF infusion in Ink4a/Arf deficient brain induces a massive proliferative response and rapid induction of early glial markers in a gradient-dependent manner.

Together, these observations provide insights into the origins, evolution, and pathophysiology of GBM and into the roles and interactions of three signature lesions, EGFR activation and loss of p16^{INK4a} and p14^{ARF}. The data presented herein provide evidence that the biological behavior of the high-grade gliomas depends not on the glial state of differentiation, but rather on the dysregulation of specific genetic elements.

Methods and Materials

Morphometric analysis of cerebral cortex and cell counts. Five coronal brain sections per animal were evaluated at 500 μm intervals from 0.5 to −2.00 with respect to Bregma according to stereotaxis atlas of the mouse brain (Franklin and Paxinos, The mouse brain in stereotaxic coordinates, San Diego, Academic Press, 1997). The thickness of the S2 somatosensory cortex as well as the horizontal distance from left to right corpus callosum at mid dorsal to ventral level were measured. To estimate the number of astrocytes in the brain, GFAP positive cells were counted in every 10th section (5 μm) under 20× objective. For the cerebral cortex, the regions containing motor and somatosensory cortex were chosen. For the hippocampus, the number of GFAP positive cells under 20× in an area covering the CA1 and the dentate gyrus was chosen.

NSC and astrocyte culture techniques. Primary NSCs were isolated from the brain subventricular zone of E13.5 embryos as described (Reynolds and Weiss, Science, 255:1707-1710 (1992)). Single cells were cultured in DMEM/F12 containing insulin/transferrin (Gibco), Penicillin/Streptomycin (Gibco), and EGF (Gibco, 20 ng/mL). Primary neurospheres were passaged by dissociation of the spheres into single cells using trituration through a fire polished pipette. Neurospheres were differentiated into secondary astrocytes by growth in 10% FBS in the media and into neurons by growth in defined media containing brain derived growth factor (BDNF, 50 ng/mL; Reynolds and Weiss, Dev. Biol., 175:1-13 (1996)). Primary astrocytes were isolated from 1 day old pups and prepared according to published methods McCarthy and de Vellis, J. Cell Biol., 85:890-902 (1980)). Cells were maintained in DMEM containing 10% FBS (GIBCO). In serial passage experiments of astrocytes and NSCs, cells were seeded at 900,000 cells per dish in 10 cm plates, and then split, counted, and reseeded every 5 days. Population doublings per passage was determined as log₂ (cells counted after 5 days in culture/cells seeded). Both primary and secondary astrocytes were analyzed for immortalization and dedifferentiation, but no difference was noted between cells from either source and results were pooled in this analysis. The time course of dedifferentiation was determined by measuring the progressive change in morphology of the cells from flat polygonal cells to final culture containing bipolar cells and neurospheres. Six independently derived cultures of each genotype were studied in the dedifferentiation experiments.

Retroviral vector and constructs. PLGIP vector was kindly provided by Dr. J. Z. Zhang (The University of Kentucky, Lexington, Ky.). The vector was modified by replacing the gfp gene with the polylinker (BamHI-Eco109I) of pBlueScript SK II (Stratagene, Calif.), designated as pMJ709. EGFR or the constitutively active mutant (vIIEGFR, lacking the exons 2-7), designated herein as EGFR*, was kindly provided by Dr. Webster Cavenee. To construct retroviruses expressing EGFR* or WT human EGFR, cDNA inserts were excised from tetop-EGFR*-KS or pPCIBA-hEGFR (H. Nakagawa, Univ. of Pennsylvania), respectively, and inserted into the BamHI-NotI sites.

Production of retrovirus stock. 293T cells (4-6×10⁶) were plated onto 10 cm dishes 14-18 hours before the transfection. Transfection by Lipofectamine Plus (Invitrogen) was performed according to the manufacturer's protocol. The retroviral supernatant was harvested 36-48 hours post transfection and used to infect target cells. Inoculated cells were selected with 2 μg/mL Puromycin for 4 days.

Stereotactic injection of cell lines. Cells for injection were suspended in Hanks Buffered Salt Solution (10,000 cells/μL) and placed on ice. Six week old SCID mice were anesthetized with ketamine (60 mg/kg) and xylazine (7.5 mg/kg) and placed in the stereotactic frame using ear bars. A hole was bored in the skull 0.5 mm anterior and 3.0 mm lateral to the Bregma. Two μL of the cell suspension were injected into the right caudate nucleus 3-5 mm below the surface of the brain using a 26 gauge needle. The scalp was closed with 5.0 silk suture. Animals were followed daily for development of neurological deficits.

Magnetic resonance imaging. Mice with neurological deficits were anesthetized with ketamine and xylazine, as above. MR imaging was performed with a 1.5-T superconducting magnet (Signa 5.0; GE Medical Systems, Milwaukee, Wis.) by using a 1.5-inch surface coil. Conventional T1-weighted (300/11, repetition time msec/echo time msec), T2-weighted (2,000/102) spin-echo images were obtained in axial, coronal, and sagittal directions. The section thickness was 1.5 mm, with a field of view of 6 cm², a 256×256 matrix, yielding a spatial resolution of 234×234×1500 microns. Gd-DTPA (Magnevist: Gadopentetate dimeglumine, Shering, Berlin, Germany) enhanced T1-weighted images were obtained 5 minutes after tail vein injection of 10 μL of Magnevist.

Brain sectioning, pathological analysis, and immunohistochemistry. For pathological analysis, brains were fixed in 10% formaldehyde for 12 hours and processed for hematoxylin and eosin (H&E) by standard techniques. The entire brain was sectioned in 1-2 mm coronal blocks and submitted in one cassette for paraffin embedding to facilitate analysis of the whole brain. For immunohistochemical analysis, sections were prepared for staining with nestin (Pharmingen), GFAP (DAKO), Olig2 (Takebayashi), and Sox10 (Rowitch) by standard techniques. For immunohistochemical analysis of cells in culture, cells were fixed in 4% paraformaldehyde for 1 hour at 4° and stained with nestin, GFAP, A2B5 (Chemicon) according to standard protocols.

Western blotting. Protein lysates from NSCs and astrocytes (1-2×10⁶) were prepared as previously described (Sharpless et al., Nature, 413:86-91 (2001)). Forty micrograms of lysate were resolved on 4-12% polyacrylamide gels (Novex) in MOPS buffer. Membranes were blotted for p16^{INK4a}, p19^{ARF}, tubulin as described elsewhere (Sharpless et al., Nature, 413:86-91 (2001)). Other antibodies used (Santa Cruz) included EGFR, EGFR-phospho, Akt, phospho-Akt, MAPK, and phospho MAPK at dilutions recommended by manufacturers.

Implantation of mini-osmotic pumps into mouse brain. EGF was unilaterally infused directly into the lateral ventricles (coordinates 4.0 mm anterior to the lambda suture, 0.7 mm lateral of midline, and 2.5 mm below the dura) using a mini-osmotic pump (Model #1007, Alza, Palo Alto, Calif.) attached to a 30 gauge cannula. EGF was infused for 7 consecutive days at a flow rate of 0.5 μL/hour with an initial pump concentration of 33 μg/mL in 0.9% saline containing 1 mg/mL mouse serum albumin (Sigma, St. Louis, Mo.).

### Example 2

Isolation of Liver Specific Stem Cells

Liver regeneration is achieved primarily by cell division of mature adult hepatocytes. When proliferation of mature hepatocytes is suppressed, facultative stem cells emerge which proliferate and differentiate into mature hepatocytes. Hepatic stem cells were obtained from E10.5 embryonic liver. Alternatively, hepatic stem cells were obtained by dedifferentiating mature hepatocytes by growth in defined media containing hepatic growth factor (HCG) and EGF. Under these conditions, mature hepatocytes lost expression of the characteristic marker polypeptides, albumin and cytochrome IIB1, and begin to express immature marker polypeptides including cytokeratin 14 and 19. These dedifferentiated hepatocytes can be redifferentiated into mature hepatocytes in vitro in the presence of matrigel (a commercial derivative of matrix extracted from EHS mouse sarcoma containing high concentrations of laminin, type VI collagen, and TGFβ1 (Fausto et al., Proc. Soc. Exp. Biol. Med. 204:237-241 (1993) and Kleinman et al., Biochem., 23: 6188-93 (1982)).

To isolate hepatic stem cells from fetal liver, E10.5 mouse embryos were used. Fetal livers were isolated, finely minced in ice cold calcium-free Hepes Buffered Salt Solution (HBSS). Pelleted fetal liver fragments were treated with collagenase D (0.2% w/v in PBS) for 20 minutes at 37° C., then with trypsin (0.05%) for 5 minutes at 37° C. and Dnase (0.09%) for 15 minutes at 37° C. The reaction was stopped by adding 3 volumes of DMEM containing 10% bovine serum. The cell suspension was filtered through a 45 μm nylon mesh, and the cells collected by centrifugation (450 g×3 minutes) and washed twice with HBSS containing 0.1% BSA. Isolated stem cells from wildtype mice or mice deficient in Ink4a/Arf−/− or p53−/− were grown in defined media containing HGF (50 ng/mL) and EGF (50 ng/mL). Following expansion in culture for 48 hours, cells were infected with VSV retrovirus carrying EGFR*-IRES-GFP or EGFR*-IRES-LacZ constructs to enable detection of donor cells following orthotopic transplantation into SCID mice.

### Example 3

Liver Cancer Models

To create liver cancer models, hepatocytes are cultured from mice harboring germline mutations in nucleic acid sequences relevant to hepatocellular carcinoma leading to loss or overexpression of the encoded polypeptides. In culture, cells are maintained as mature hepatocytes or dedifferentiated into hepatic stem cells as described in Example 2. To generate cancer, cells are injected into recipient mice either hematogenously or through direct inoculation of a specific tissue (e.g., into the liver, lung, or brain). Then, animals are monitored for cancer growth as described herein.

### Example 4

Isolation of Pancreas Specific Stem Cells

Pancreatic stem cells were isolated from adult mouse pancreas using a two step protease digestion method. Harvested tissue were chopped into small pieces and digested in Collagenase D (2.5 mg/mL in HBSS) for 25 minutes at 37° C. Cells were then pelleted, washed with calcium-free PBS and further digested with 3 volumes of DMEM containing 10% bovine serum. Cell viability was determined by counting cells using trypan blue exclusion in a hemocytometer. Once isolated, pancreatic stem cells were maintained in defined media containing bFGF (20 ng/mL) and EGF (20 ng/mL).

### Example 5

Pancreatic Cancer Models

To generate pancreatic cancer, pancreatic stem cells from mice deficient in p53 and/or Ink4a/Arf and overexpressing TGFα or EGF under the elastase promoter are injected orthopically into the subcutaneous space, under the renal capsule, or into the body of the pancreas of the adult SCID mouse. As described herein, mice are monitored for the generation of cancer over a 3 month period.

### OTHER EMBODIMENTS

It is to be understood that while the invention has been described in conjunction with the detailed description thereof, the foregoing description is intended to illustrate and not limit the scope of the invention. Other aspects, advantages, and modifications are within the scope of the invention.

What is claimed is:

1. A non-human animal comprising tumor cells originating, in said animal, from at least one heterologous stem cell, wherein said at least one heterologous stem cell comprises a genetic alteration.

2. The animal of claim 1, wherein said animal is a rodent.

3. The animal of claim 1, wherein said animal is a mouse.

4. The animal of claim 1, wherein said animal is immunocompromised.

5. The animal of claim 1, wherein said animal is a SCID mouse.

6. The animal of claim 1, wherein said at least one heterologous stem cell is a mouse cell or a human cell.

7. The animal of claim 1, wherein said at least one heterologous stem cell is a neural stem cell.

8. The animal of claim 1, wherein said at least one heterologous stem cell is an embryonic stem cell or tissue-specific stem cell.

9. The animal of claim 1, wherein said at least one heterologous stem cell is a dedifferentiated cell.

10. The animal of claim 9, wherein said dedifferentiated cell is a dedifferentiated astrocyte, dedifferentiated hepatocyte, or dedifferentiated pancreatic cell.

11. The animal of claim 9, wherein said dedifferentiated cell was produced from a cultured cell.

12. The animal of claim 11, wherein said cultured cell is selected from the group consisting of astrocytes, melanocytes, pancreatic cells, mammary epithelial cells, and hepatocytes.

13. The animal of claim 1, wherein said genetic alteration comprises an introduced nucleic acid molecule.

14. The animal of claim 13, wherein said introduced nucleic acid molecule encodes a polypeptide.

15. The animal of claim 14, wherein said polypeptide is an oncogene product.

16. The animal of claim 14, wherein said polypeptide is selected from the group consisting of H-RAS, epidermal growth factor receptor, ErbB2, and MDM2.

17. The animal of claim 14, wherein said polypeptide is expressed by said at least one heterologous stem cell.

18. The animal of claim 1, wherein said genetic alteration comprises a mutation.

19. The animal of claim 18, wherein said mutation is in a tumor suppressor gene.

20. The animal of claim 19, wherein said tumor suppressor gene encodes a polypeptide selected from the group consisting of p16^{INK4a}, p19^{ARF}, PTEN, and pRB.

21. The animal of claim 20, wherein said genetic alteration further comprises an introduced nucleic acid molecule.

22. The animal of claim 20, wherein said at least one heterologous stem cell has reduced expression of said tumor suppressor gene.

23. The animal of claim 19, wherein said at least one heterologous stem cell has reduced tumor suppressor activity.

24. The animal of claim 1, wherein said tumor cell is a pancreatic tumor cell, a liver tumor cell, a breast tumor cell, or a brain tumor cell.

25. An isolated stem cell, wherein said isolated stem cell is INK4a/ARF^{-/-}.

26. A non-human animal comprising at least one heterologous stem cell, wherein said at least one heterologous stem cell is tumorigenic.

27. A method for making a chimeric non-human animal, said method comprising:
(a) genetically altering stem cells, and
(b) introducing said stem cells into a non-human animal thereby forming said chimeric non-human animal, wherein said chimeric non-human animal develops tumor cells from at least one of said stem cells.

28. A method for making a cancer model, said method comprising:
(a) genetically altering stem cells to have reduced tumor suppressor gene expression or increased oncogene expression,
(b) introducing said stem cells into at least one non-human animals, and
(c) identifying a cancerous non-human animal from said at least one non-human animals, wherein said cancerous non-human animal comprises a tumor cell originating from at least one of said introduced stem cells, said cancerous non-human animal being said cancer model.

29. A method for identifying an agent that reduces cancer in a non-human animal, said method comprising:
(a) administering a test agent to said animal, wherein said animal comprises tumor cells originating, in said animal, from at least one heterologous stem cell, and
(b) determining whether or not said test agent reduces the number of said tumor cells within said animal, wherein a reduction in the number of said tumor cells within said animal indicates that said test agent is said agent.
